(19) United States
(12) Patent Application Publication
(10) Pub. No.: US 2020/0265922 A1
(43) Pub. Date: Aug. 20, 2020

(54) Comprehensive Genomic Transcriptomic Tumor-Normal Gene Panel Analysis For Enhanced Precision In Patients With Cancer

(71) Applicant: NANTOMICS, LLC, Culver City, CA (US)

(72) Inventors: Sharooz Rabizadeh, Culver City, CA (US); Chad Garner, Culver City, CA (US); Rahul Parulkar, Culver City, CA (US); Christopher Szeto, Culver City (US)

(21) Appl. No.: 16/754,727

(22) PCT Filed: Oct. 9, 2018
(86) PCT No.: PCT/US2018/055025

§ 371 Date: Apr. 8, 2020

Related U.S. Application Data
(60) Provisional application No. 62/618,893, filed on Jan. 18, 2018.
(60) Provisional application No. 62/570,580, filed on Oct. 10, 2017.

(51) Int. Cl. G16B 20/20; G16B 30/10; G16B 45/00

(52) U.S. Cl. CPC G16B 20/20; G16B 45/00; G16B 30/10

(57) ABSTRACT

Improved accuracy of SNV-based genetic tests is performed using DNA sequencing data from a tumor sample and a matched normal sample to determine SNVs, and RNA sequencing data from the tumor sample are used to ascertain expression of so identified SNVs.

This application claims priority to our U.S. Provisional Patent Application Ser. No. 62/570,580, which was filed Oct. 10, 2017, and US provisional application with the Ser. No. 62/618,893, which was filed Jan. 18, 2018, both of which are incorporated herein by reference in their entireties.

### FIELD OF THE INVENTION

The field of the invention is profiling of omics data as they relate to cancer, especially as it relates to the reduction of false positive results for polymorphisms in gene panel tumor-only analysis for various cancers.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Commercially-available clinical-grade gene panel tests, based on DNA sequencing are widely used in clinical practice. These panel-based tests, based on tumor-only analysis are presently the most common approach for genomic testing in oncology for clinical decision support. Sequencing-based approaches seek to identify the somatically-derived genomic variation that drives tumor growth and to precisely distinguish these genetic variants from the large background of inherited germline genomic variation that inevitably predominates in the tumor genome.

In 2016, the Centers for Medicare and Medicaid Services (CMS) authorized coverage of a tumor-only DNA sequencing-based test of 35 genes that were intended to inform lung cancer treatment. This currently CMS approved test is based on tumor-only analysis of a targeted gene panel, with the specific exclusion of comparing such analysis to the patient's normal germline tissue. Instead the current approved test utilizes a reference genome and filtration technique to distinguish ‘true’ somatic variants from either normal polymorphism or inherited germline variants. The test (MolDX: L36194) is defined as a “single test using tumor tissue only (i.e., not matched tumor and normal) that does not distinguish between somatic and germline alterations”. However, this tumor-only approach has been reported by others to increase the risk of mistakenly identifying germline mutations as somatically-derived genetic changes and potential cancer driver mutations (“false positives”). While it was recently shown that false positive rates associated with tumor-only sequencing can at least to some degree be reduced by molecular pathologist review of all putative somatic variants, such individual review is generally time consuming and still error prone.

Thus, there remains a need for improved methods of analyzing omics data from cancer patients, especially where false positive test results are likely.

### SUMMARY OF THE INVENTION

The inventive subject matter is directed to various methods of analyzing and/or identifying tumor-associated single nucleotide variants (SNVs) using genomics and transcriptomics data of tumor DNA, germline DNA, and tumor RNA from a patient, which unexpectedly improves accuracy, and with that, chances of effective treatment.

Thus, in one aspect of the inventive subject matter, the inventors contemplate a method of performing a SNV-based cancer test with increased accuracy. This method includes a step of obtaining DNA sequencing data from a tumor sample and a matched normal sample (i.e., non-tumor sample of the same patient), and a further step of obtaining RNA sequencing data from the tumor sample. The method then further includes a step of determining presence of DNA single nucleotide variants in the tumor sample relative to the matched normal sample and a step of determining expression of the DNA single nucleotide variants using the RNA sequencing data. In some embodiments, the step of determining the presence of the DNA single nucleotide variant is performed using location guided synchronous alignment of the DNA sequencing data from the tumor sample and the matched normal sample. Preferably, the method further includes a step of identifying at least one DNA single nucleotide variant as being associated with cancer status of the patient based on the presence and the expression of the single nucleotide variants.

Most typically, the DNA sequencing data is whole genome DNA sequencing data. Preferably, DNA sequencing data of the tumor tissue have a read depth of at least 50×, and/or the DNA sequencing data of the matched normal tissue have a read depth of at least 30×. In some embodiments, the method further comprises a step of filtering the DNA single nucleotide variants using allele frequencies of the DNA single nucleotide variants.

In another aspect of the inventive subject matter, the inventors contemplate a method of identifying a treatment option for a patient with increased accuracy. This method includes a step of determining presence of DNA single nucleotide variants in the tumor sample relative to the matched normal sample of the patient, and a step of determining expression of the DNA single nucleotide variants using the RNA sequencing data. Then, the method further comprises a step of identifying the treatment option targeting a gene having at least one DNA single nucleotide variant that is expressed as RNA.

Preferably, the step of determining the presence of the DNA single nucleotide variant is performed using location guided synchronous alignment of the DNA sequencing data from the tumor sample and the matched normal sample. In some embodiments, the step of determining the presence of the DNA single nucleotide variant is performed using an in silico gene panel having a plurality of reference sequences of tumor associated genes. In such embodiment, it is preferred that the in silico gene panel is cancer type-specific and/or the tumor associated genes are selected from a group consisting of ABL1, EGFR, GNAS, KRAS, PTPN11, AKT1, ERBB2, GNAQ, MET, RB1, ALK, ERBB4, HNF1A, MLH1, RET, APC, EZH2, HRAS, MPL, SMAD4, ATM, FBXW7, IDH1, NOTCH1, SMARCB1, BRAF, FGFR1, JAK2, NPM1, SMO, CDH1, FGFR2, JAK3, NRAS, SRC, CDKN2A, FGFR3, IDH2, PDGFRA, STK11, CSF1R, FLT3, KDR, PIK3CA, TP53, CTNNB1, GNA11, KIT, PTEN, VHL.

In some embodiments, the method further comprises a step of filtering the DNA single nucleotide variants using allele frequencies of the DNA single nucleotide variants.

In some embodiments, the step of determining the expression of the DNA single nucleotide variants comprises measuring RNA expression level of the DNA single nucleotide variants and comparing with a predetermined threshold. In such embodiment, it is contemplated that the method may further comprise a step of ranking the DNA single nucleotide variants based on the RNA expression level and/or a step of classifying the DNA single nucleotide variants into an “expressed” or “non-expressed” group based on the comparison with the predetermined threshold.

In still another aspect of the inventive subject matter, the inventors contemplate a method of testing a patient sample that includes a step of generating or obtaining DNA omics data from tumor and matched normal tissue of the patient, and a further step of generating or obtaining RNA omics data from the tumor tissue of the patient. In yet another step, tumor and patient specific SNVs are identified in the DNA omics data of the tumor using the DNA omics data of the matched normal tissue, and the RNA omics data from the tumor tissue are used to confirm presence and quantity of expression of the SNV.

Preferably, the DNA and/or RNA omics data are in BAM format, and the step of identifying tumor and patient specific SNVs is performed using incremental synchronous alignment (e.g., using BAMBAM, which may use the DNA omics data and the RNA omics data). Most typically, but not necessarily, the RNA omics data are RNAseq data, and/or the SNVs in the DNA omics data of the tumor are in a cancer driver gene or in an inherited cancer risk gene. For example, suitable cancer driver genes include ACT1, ACT2, ACT3, APC, ATM, BRAF, BRCA1, BRCA2, CHEK1, CHEK2, EGFR, ERBB2, ERBB3, ERBB4, FGFR1, FGFR2, FGFR3, HRAS, JAK3, KIT, KRAS, MET, NOTCH1, NRAS, PALB2, PDGFRA, PIC3CA, PTEN, SMO, SRC, and TP53, and suitable inherited cancer risk genes include APC, ATM, AXIN2, BMPR1ACHD1, CHEK2, EPCAM, GREM1, MLH1, MSH2, MSH6, MUTYH, PMS2, POLD1, POLE, PTEN, SMAD4, STK11, and TP53.

In still another aspect of the inventive subject matter, the inventors contemplate a method of increasing accuracy in identifying a true somatic single nucleotide in a patient having a tumor. This method includes steps of obtaining DNA sequencing data from a tumor sample and a matched normal sample of a patient, and further obtaining RNA sequencing data from the tumor sample, determining presence of DNA single nucleotide variants in the tumor sample relative to the matched normal sample, determining presence of DNA single nucleotide variants in the tumor sample relative to the matched normal sample, and identifying at least one DNA single nucleotide variant as being associated with cancer status of the patient based on the presence and the expression of the single nucleotide variants.

Most typically, the DNA sequencing data is whole genome DNA sequencing data. In some embodiments, the DNA sequencing data of the tumor tissue have a read depth of at least 50×, and/or the DNA sequencing data of the matched normal tissue have a read depth of at least 30×.

In some embodiments, the step of determining the presence of the DNA single nucleotide variant is performed using location guided synchronous alignment of the DNA sequencing data from the tumor sample and the matched normal sample. In other embodiments, the method may further comprise a step of filtering the DNA single nucleotide variants using allele frequencies of the DNA single nucleotide variants.

In some embodiments, the step of determining the presence of the DNA single nucleotide variant is performed using an in silico gene panel having a plurality of reference sequences of tumor associated genes. In such embodiments, it is preferred that the in silico gene panel is cancer type-specific, and/or the tumor associated genes are selected from a group consisting of ABL1, EGFR, GNAS, KRAS, PTPN11, AKT1, ERBB2, GNAQ, MET, RB1, ALK, ERBB4, HNF1A, MLH1, RET, APC, EZH2, HRAS, MPL, SMAD4, ATM, FBXW7, IDH1, NOTCH1, SMARCB1, BRAF, FGFR1, JAK2, NPM1, SMO, CDH1, FGFR2, JAK3, NRAS, SRC, CDKN2A, FGFR3, IDH2, PDGFRA, STK11, CSF1R, FLT3, KDR, PIK3CA, TP53, CTNNB1, GNA11, KIT, PTEN, VHL.

In some embodiments, the step of determining the expression of the DNA single nucleotide variants comprises measuring RNA expression level of the DNA single nucleotide variants and comparing with a predetermined threshold. In such embodiments, it is also contemplated that the method may further comprise a step of ranking the DNA single nucleotide variants based on the RNA expression level, and/or classifying the DNA single nucleotide variants into an “expressed group” or a “non-expressed group” based on the comparison with the predetermined threshold.

Various objects, features, aspects and advantages of the inventive subject matter will become more apparent from the following detailed description of preferred embodiments, along with the accompanying drawings.

### BRIEF DESCRIPTION OF THE DRAWING

FIG. 1 is a graph depicting the number of false positive results that would occur among 45 lung cancer patients tested in Example 1.

FIG. 2 is a graph depicting the number of false positive results that would occur among all cancer patients tested in Example 1.

FIG. 3 is a graph depicting the number of true positive and false positive SNVs for the 45 lung cancer patients tested in Example 1.

FIG. 4 is a graph depicting the number of true positive and false positive SNVs for all cancer patients tested in Example 1.

FIGS. 5A-5B are graphs depicting the number of somatic and germline origin of SNVs identified by gastro-intestinal cancer patients in Example 2

FIGS. 6A-6B are graphs depicting the number of true positive and false positive SNVs filtered with allele frequencies by genes in Example 2.

FIG. 7 is a graph depicting the number of true positive and false positive SNVs filtered with allele frequencies by patients in Example 2.

FIG. 8 is a graph depicting the number of true positive and false positive SNVs in gastro-intestinal cancer patients identified by RNA expression analysis in Example 2.

FIG. 9 is a graph depicting the number of tumor samples that were analyzed for genomics and/or transcriptomics data by types of tumor in Example 3.

FIG. 10 is a graph depicting the somatic and germline origin of SNVs identified in various types of cancer patients in Example 3.

FIG. 11 is a graph depicting the true positive and false positive SNVs filtered with allele frequencies in Example 3.

FIG. 12 is a graph depicting the number of missense/nonsense SNVs that are expressed or not expressed in Example 3.

FIG. 13 is a graph depicting the number of somatic SNVs that are expressed or not expressed in Example 3.

### DETAILED DESCRIPTION

The inventors have unexpectedly discovered that single nucleotide variants (SNVs) identified by conventional tumor DNA analysis poses high risk of including false-positive and/or false-negative SNVs as majority of such SNVs identified are germline-originated variants. The inventors further discovered that many of identified somatic SNVs are not expressed as RNA such that identification of such non-expressed somatic SNVs as molecular target for tumor treatment leads to ineffective cancer treatment. Viewed from the different perspective, the inventors now have discovered that the accuracy of a single nucleotide variant-based cancer test can be significantly increased by simultaneous bioinformatics analysis of tumor genomic DNA relative to matched normal to identify somatic SNVs and of tumor RNA expression to identify expressed or nonexpressed somatic SNVs. Consequently, the inventors contemplate that such identified somatic SNVs that is expressed in the tumor can be associated with cancer status, and further be identified as an effective target of the tumor treatment.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

Thus, in one especially preferred aspect of the inventive subject matter, the inventors contemplate that an accuracy of a single nucleotide variant-based cancer test can be significantly increased by obtaining DNA and RNA data from a tumor sample and/or a matched normal sample of a patient to so determine DNA single nucleotide variants in the tumor sample relative to the matched normal sample and determine expression of the DNA single nucleotide variants. It is contemplated that DNA single nucleotide variants that is expressed as RNA can be associated with cancer status of the patient with high accuracy.

### Obtaining Omics Data

Any suitable methods of obtaining a tumor sample (tumor cells or tumor tissue) from the patient (or healthy tissue from a patient or a healthy individual as a comparison) are contemplated. Most typically, a tumor sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, the tumor cells or tumor tissue may be fresh or frozen. For other example, the tumor cells or tumor tissues may be in a form of cell/tissue extracts. In some embodiments, the tumor samples may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. Preferably, a healthy tissue of the patient or matched normal tissue (e.g., patient's non-cancerous breast tissue) can be obtained or a healthy tissue from a healthy individual (other than the patient) can be also obtained via a similar manner as a comparison.

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor cells or tumor tissue, DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or healthy tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

### DNA Single Nucleotide Variants in the Tumor Sample Relative to the Matched Normal Sample

It is contemplated that somatic SNVs can be distinguished and identified from germline SNVs by comparing the genomic DNA sequences obtained from tumor tissue and matched normal tissue of a patient (e.g., non-tumor tissue of a patient including liquid biopsy of nontumor blood sample). With respect to the analysis of tumor and matched normal tissue of a patient, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19) or sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, omics data analysis may reveal missense and nonsense mutations, changes in copy number, loss of heterozygosity, deletions, insertions, inversions, translocations, changes in microsatellites, etc.

Moreover, it should be noted that the data sets are preferably reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

In some embodiments, where the whole genome or exome sequencing data of the tumor and matched normal is compared with external reference sequences, it is contemplated that the external reference sequences are organized as an in silico gene panel. Preferably, the in silico gene panel includes a plurality of tumor-associated genes, including tumor-driver gene(s) or cancer-driver gene(s) (e.g., EGFR, KRAS, TP53, APC, etc.) and/or drug-sensitivity or metabolism related genes. It is contemplated that the numbers and types of genes in the in silico gene panel may vary depending on the type of cancer the patient may have or be diagnosed (e.g., cancer type-specific in silico gene panel), and preferably includes at least 20 genes, at least 30 genes, at least 40 genes, or at least 50 genes. For example, the in silico gene panel may include whole genome sequences and/or whole exome sequences of ABL1, EGFR, GNAS, KRAS, PTPN11, AKT1, ERBB2, GNAQ, MET, RB1, ALK, ERBB4, HNF1A, MLH1, RET, APC, EZH2, HRAS, MPL, SMAD4, ATM, FBXW7, IDH1, NOTCH1, SMARCB1, BRAF, FGFR1, JAK2, NPM1, SMO, CDH1, FGFR2, JAK3, NRAS, SRC, CDKN2A, FGFR3, IDH2, PDGFRA, STK11, CSF1R, FLT3, KDR, PIK3CA, TP53, CTNNB1, GNA11, KIT, PTEN, VHL.

Additionally, it is also contemplated that such identified DNA single nucleotide variants are further filtered using DNA allele frequencies (e.g., using a public database with reported population allele frequencies). In some embodiments, the DNA single nucleotide variants can be filtered with a predetermined frequency threshold, for example, reported allele frequencies ≥0.01 (1%), preferably ≥0.005 (0.5%), or more preferably ≥0.001 (0.1%).

Additionally, the significance of the sequence change (DNA single nucleotide variants) can be assessed by variant calling where the genomics data is in BAM file format. Because BamBam keeps the sequence data in the pair of files in sync across the genome, a complex mutation model that requires sequencing data from both BAM files derived from two biological samples as well as the reference can be implemented easily. This model aims to maximize the joint probability of both sequence strings of two biological samples. To find the optimal genotypes of two sequence strings from two biological samples, the inventors aim to maximize the likelihood defined by:

P(D_{g},Dₜ,G_{g},Gₜ|α,r)=P(D_{g}|G_{g})P(G_{g}|r)P(Dₜ|G_{g},Gₜ,α)P(Gₜ|G_{g}) (1)

P(D_{↓g},D_{↓t},G_{↓g},G_{↓t}┤|α,r)=P(D_{↓g}┤|G_{↓g})P(G_{↓g}┤|r)P(D_{↓t}┤|G_{↓g},G_{↓t},α)P(G_{↓t}┤|G_{↓g}) (1)

where r is the observed reference allele, α the fraction of normal contamination, and the genotypes of sequence string 1 and 2 are defined by Gt=(t₁, t₂) and Gg=(g₁, g₂), respectively, where t₁, t₂, g₁, g₂ε{A, T, C, G}. The sequence data of sequence string 1 and 2 are defined as a set of reads Dₜ={dₜ¹, dₜ², . . . , dₜ^{m}} and D_{g}={d_{g}¹, d_{g}², . . . , d_{g}^{m}}, respectively, with the observed bases dₜⁱ, d_{g}ⁱε{A, T, C, G}. All data used in the model must exceed user-defined base and mapping quality thresholds.

The probability of the germline alleles given the germline genotype is modeled as a multinomial over the four nucleotides:

$$P ( {D}_{g} \vert {G}_{g} ) = \frac{n !}{{n}_{A} ! {n}_{T} ! {n}_{G} ! {n}_{C} !} {Π}_{j}^{n} P ( {d}_{g}^{i} \vert {G}_{g} ) ,$$

where n is the total number of germline reads at this position and n_{A}, n_{G}, n_{C}, n_{T} are the reads supporting each observed allele. The base probabilities, P(d_{g}ⁱ|G_{g}), are assumed to be independent, coming from either of the two parental alleles represented by the genotype G_{g}, while also incorporating the approximate base error rate of the sequencer. The prior on the sequence string 1 genotype is conditioned on the reference base as:

P(G_{g}|r=a)={μₐₐ,μ_{ab},μ_{bb}}

where μₐₐ is the probability that the position is homozygous reference, μ_{ab} is heterozygous reference, and μ_{bb} is homozygous non-reference. At this time, the sequence string 1prior does not incorporate any information on known, inherited SNPs.

The probability of the set of sequence 2 reads is again defined as multinomial

$$P ( {D}_{t} \vert {D}_{t} , {G}_{g} , α ) = \frac{n !}{{n}_{A} ! {n}_{T} ! {n}_{G} ! {n}_{C} !} {Π}_{j}^{n} P ( {d}_{g}^{i} \vert {G}_{t} , {G}_{g} , α ) ,$$

where m is the total number of germline reads at this position and m_{A}, m_{G}, m_{C}, m_{T} are the reads supporting each observed allele in the sequence 2 dataset, and the probability of each sequence 2read is a mixture of base probabilities derived from both sequence 2 and sequence 1 genotypes that is controlled by the fraction of normal contamination, α, as

P(dₜⁱ|Gₜ,G_{g}α)=αP(dₜⁱ|Gₜ)+(1−α)P(dₜⁱ|G_{g})

and the probability of the sequence 2 genotype is defined by a simple mutation model from on the sequence 1 genotype

P(Gₜ|G_{g})=max[P(t₁|g₁)P(t₂|g₂),P(t₁|g₂)P(t₂|g₁)],

where the probability of no mutation (for example, t1=g1) is maximal and the probability of transitions (that is, A→G,T→C) are four times more likely than transversions (that is, A→T,T→G). All model parameters, α, μaa, μab, μbb, and base probabilities, P(di|G), for the multinomial distributions are user-definable.

The sequence 2 and 1 genotypes, Gt max, Gg maxi, selected are those that maximize (1), and the posterior probability defined by

$$\frac{P ( {D}_{g} , {D}_{t} , {G}_{g}^{max} , {G}_{t}^{max} \vert α , r )}{{\sum }_{i , j} P ( {D}_{g} , {D}_{t} , {G}_{g} = i , {G}_{t} = j \vert α , r )}$$

can be used to score the confidence in the pair of inferred genotypes. If the sequence 2 and sequence lgenotypes differ, the mutations in sequence 2 will be reported along with its respective confidence.

Maximizing the likelihood of one or both sequence 1 and 2 genotypes helps to improve the accuracy of both inferred genotypes, especially in situations where one or both sequence datasets have low coverage of a particular genomic position. Other mutation calling algorithms, such as MAQ and SNVMix, that analyze a single sequencing dataset are more likely to make mistakes when the non-reference or mutant alleles have low support (Li, H., et al. (2008) Mapping short DNA sequencing reads and calling variants using mapping quality scores, Genome Research, 11, 1851-1858; Goya, R. et al. (2010) SNVMix: predicting single nucleotide variants from next-generation sequencing of tumors, Bioinformatics, 26, 730-736).

In addition to collecting allele support from all reads at a given genomic position, information on the reads are collected (such as which strand, forward or reverse, the read maps to, the position of the allele within the read, the average quality of the alleles, etc.) and used to selectively filter out false positive calls. We expect a random distribution of strands and allele positions for all of the allele supporting a variant, and if the distribution is skewed significantly from this random distribution (that is, all variant alleles are found near the tail end of a read), then this suggest that the variant call is suspect.

It is also contemplated that the variant calling for sequence changes can be also performed by other analysis tools, including, but not limited to, MuTect (Nat Biotechnol. 2013 March; 31(3):213-9), MuTect2, HaploTypeCaller, Strelka2 (Bioinformatics, Volume 28, Issue 14, 15 Jul. 2012, Pages 1811-1817), or other genomic artifact detection tool.

### Expression of the DNA Single Nucleotide Variants

In addition, omics data of tumor and/or matched normal comprises transcriptome data set that includes sequence information and expression level (including expression profiling or splice variant analysis) of RNA(s) (preferably cellular mRNAs) that is obtained from the patient. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers, are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

The inventors contemplate that in some embodiments, the expression of the gene (or a portion of a gene) having one or more single nucleotide variant(s) can be determined by RNA sequencing data (e.g., RNAseq). In such embodiments, the expression of the one or more single nucleotide variant(s) can be assessed as presence or absence (or existence or non-existence) of the one or more single nucleotide variant(s) in the expressed RNA. Consequently, based on the RNA sequencing data the single nucleotide variant(s) can be grouped into “expressed group” or a “non-expressed group”. In other embodiments, the e expression of the gene (or a portion of a gene) having one or more single nucleotide variant(s) can be determined by combining RNAseq data and RNA quantification data (e.g., using qPCR and/or rtPCR). In such embodiments, the expression level of the one or more single nucleotide variant(s) can be assessed as presence or absence (or existence or non-existence) by comparing with a predetermined threshold. It is contemplated that the predetermined threshold may vary depending on the genes. For example, the predetermined threshold may be 10%, 5%, or 1% of the average RNA expression level of the gene in the same or similar types of tissue (e.g., liver, lung, etc.) of healthy individuals or the RNA expression level of the gene in the matched normal tissue of the patient. Alternatively, the predetermined threshold may vary depending on the qPCR and/or rtPCR noise level in the given reaction(s). For example, the predetermined threshold may be within 20%, within 10%, within 5% of the noise level of the qPCR and/or rtPCR reaction. Consequently, based on the RNA expression level, the single nucleotide variant(s) can be grouped into “expressed group” where the expression level is on or above the predetermined threshold, or a “non-expressed group” where the expression level is below the predetermined threshold.

Without wishing to be bound by any specific theory, the inventors contemplate that combination of genomics data and transcriptomics data to identify expressed DNA single nucleotide variants significantly reduce false-positive rate (mistakenly identifying germline mutations as somatically-derived cancer driver mutations, and/or identifying somatically-derived cancer driver mutations that are not expressed as an effective mutation, etc.) and/or false-negative rate (e.g., true tumor somatic SNVs are excluded, etc.). Reduction in false-positive and/or false-negative rate in identification of DNA single nucleotide variants in tumor-associated genes further significantly increases the efficiency and accuracy in identifying the genes associated with tumor and/or cancer, and also in identifying any effective treatment regimen with reduced undesired side effects or toxicity as the numbers of expressed DNA single nucleotide variants to be analyzed and targeted in association with the tumor or cancer can be significantly reduced in the relatively early stage of analysis or application.

Consequently, the inventors further contemplate that based on the presence/absence and the expression of the single nucleotide variants, such single nucleotide variants can be identified as a cancer-associated variants (or mutation) that may be further associated with a cancer status of the patient. As used herein, the term “cancer status” refers any molecular, physiological, pathological condition of a cancer or a tumor. Thus, the cancer status may include an anatomical type of cancer (e.g., gastrointestinal cancer, lung cancer, brain tumor, etc.), a metastatic status of the tumor (e.g., metastasized, high-tendency of metastasis, non-metastasized, etc.), tumor clonality, an immune status of the tumor tissue (e.g., immune suppressed, immune-activated, immune-dormant, etc.), prognosis of the tumor (e.g., stage of the tumor, grade of the tumor including the morphogenesis of the tumor, etc.). In addition, the cancer status may include the sensitivity or resistance of the tumor to a tumor treatment (e.g., resistance to checkpoint inhibitor administration, sensitivity to cytokine treatment, etc.), a toxicity by a chemotherapeutic drug (e.g., due to a mutation/single nucleotide variant in an element of CYP2D6 enzyme-mediated pathway, etc.).

In some embodiments, the association of the expressed DNA single nucleotide variants to a status of tumor or cancer may be quantified by providing significance score(s). For example, the significance score can be determined by combining sub-scores for number of DNA single nucleotide variants (1 score per one nucleic acid change), the type of DNA single nucleotide variants (e.g., nonsense mutation, missense mutation, etc.), location of DNA single nucleotide variants (e.g., exon 3 of the gene encoding the functional binding domain, etc.), and physiological impact (dominant negative factor for signaling pathway B). Also, the significance score can be determined by the expression of the gene including the DNA single nucleotide variants (e.g., −1 for each non-expressed DNA single nucleotide variant, +1 for each expressed DNA single nucleotide variant, or various incremental scores based on the expression levels of gene including DNA single nucleotide variants such as 1 score per each 10% increased expression of the gene including DNA single nucleotide variants, etc.). Thus, in such embodiments, the significance of DNA single nucleotide variants can be ranked based on the expression (presence or absence in RNA) or expression level (increase or decrease of the RNA expression level compared to normal tissue or healthy individual). Alternatively and/or additionally, the significant score(s) of genes including DNA single nucleotide variants can be used to further rank the genes or DNA single nucleotide variants.

The inventors further contemplate that such identified and/or ranked DNA single nucleotide variants and/or genes including DNA single nucleotide variants can be further used to identify a treatment option to treat the cancer or tumor of the patient. For example, Upon confirmation of the DNA single nucleotide variants (identified by tumor matched-normal sequencing) in the RNA and upon confirmation of the RNA as being expressed (e.g., at least 25% as compared to matched normal, at least 50% as compared to matched normal, at least 75% as compared to matched normal, at least 100% as compared to matched normal, at least 125% as compared to matched normal, or at least 150% as compared to matched normal) in a tumor-associated gene having one or more DNA single nucleotide variants, a drug targeting the tumor-associated gene is administered to the patient in a dose and schedule effective to treat the tumor. As used herein, the drug targeting the tumor-associated gene may include a drug that modulates the expression of the gene (in transcriptional level or translational level), a drug that modulate the post-translational modification of the gene product (protein), a drug that modulate the activity of the gene product (protein), or a drug that modulate the degradation of the gene product (protein).

As used herein, the term “administering” a drug or a cancer treatment refers to both direct and indirect administration of the drug or the cancer treatment. Direct administration of the drug or the cancer treatment is typically performed by a health care professional (e.g., physician, nurse, etc.), and wherein indirect administration includes a step of providing or making available the drug or the cancer treatment to the health care professional for direct administration (e.g., via injection, oral consumption, topical application, etc.).

### Example 1

Currently approved tests for lung cancer are based on tumor-only analysis of a targeted gene panel, with the specific exclusion of patient's normal germline tissue. However, as is shown in more detail below, the tumor-only approach substantially increases the risk of mistakenly identifying germline mutations as somatically-derived cancer driver mutations (i.e., false positives), and further fails to inform a physician where a potentially druggable target is even present in meaningful quantities in the tumor.

More specifically, the inventors discovered that 94% of all variants found in a currently approved, gene panel tumor-only analysis for lung cancer patients were indeed false positive polymorphisms, and that 48% remained false positives after stringent filtration. Of true somatic mutations identified in a directly druggable subset of this panel, about 18% were not expressed, compounding the risk of inaccurate treatment decisions and treatment futility. On the backdrop of such diagnostic failure it has become apparent that there is a need for improved identification of true tumor somatic variants. As is described in more detail below, such improved analysis has been accomplished by coordinated analyses of tumor DNA, germline DNA, and tumor RNA.

Based on concerns of false positives of tumor-only gene panel analysis, the inventors sought to demonstrate enhanced precision afforded by simultaneously sequencing and analyzing both tumor and germline, and improving the confidence with which mutations can be identified as potential drivers of disease. As is discussed in more detail below, the inventors undertook a study to demonstrate that i) molecular characterization of tumors for the purpose of treatment decision support is appreciably more precise by bioinformatic analysis of using the patient's normal tissue as control, that is tumor-normal DNA sequencing and that the precision of true somatic variants so identified is further enhanced when combined with RNA sequencing, ii) bioinformatic filtration of polymorphisms from tumor-only sequence analysis does not match the precision of tumor-normal genomic analysis, iii) confirmation that any true somatic mutation is expressed in the mRNA provides the critical second line of evidence that a detected somatic tumor mutation may play a role as an oncogenic driver.

In this example, DNA sequencing of tumor and normal germline genomes of the 35-gene panel authorized for coverage by CMS from 45 lung cancer patients and 621 total cancer patients with 33 cancer types was used to quantify the rate of false positive tumor somatic variants originating from the use of the tumor-only sequencing approach. Potential increase in precision from expression analysis of alterations in these 35 genes by RNA sequencing was also assessed.

Patients and Sequencing Data:

In this example, the inventors focused on mutation analysis in 35 genes that have been previously authorized for Medicare coverage by CMS to enable clinicians to better define therapy for patients with lung cancer. CMS approved the use of this gene panel only when genomic variants were identified through tumor only DNA sequencing and analysis (i.e., not matched tumor and normal). This approach does not directly distinguish between somatic and germline alterations. The panel included 25 genes implicated as somatic tumor drivers (tumor driver gene panel) and 10 genes that are known to affect inherited cancer risk (inherited risk gene panel). The tumor driver gene panel consists of: ALK, BRAF, CDKN2A, CEBPA, DNMT3A, EGFR, ERBB2, EZH2, FLT3, IDH1, IDH2, JAK2, KIT, KMT2A, KRAS, MET, NOTCH1, NPM1, NRAS, PDGFRA, PDGFRB, PGR, PIK3CA, PTEN, RET. The inherited cancer risk panel consisted of: APC, BMPR1A, EPCAM, MLH1, MSH2, MSH6, PMS2, POLD1, POLE, STK11.

Whole genome sequencing data from tumor DNA, tumor RNA, and normal DNA of 621 cancer patients was analyzed to identify somatically-derived single nucleotide variants potentially contributing to cancer growth and expansion. This example included 45 lung cancer patients. All patients provided informed consent for the use of the data described in this study. DNA and RNA was extracted from preserved tissue and sequenced using the Illumina platform in a NantOmics Clinical Laboratory Improvement Amendments (CLIA)- and Certified Authorization Profession (CAP)-certified sequencing laboratory. Performance characteristics of the test used include >95% sensitivity and >99% specificity to detect SNVs transcribed and expressed as RNA. Normal germline and tumor genomes were sequenced to read depths of approximately 30× and 60×, respectively. Approximately 300 million RNA sequencing reads were generated for each tumor.

Data Analysis:

DNA sequencing data was aligned to GRCh37 (www.ncbi.nlm.nih.gov/assembly/2758/) by BWA, duplicate-marked by samblaster, and indel realignment and base quality recalibration performed by GATK v2.3. RNA sequencing data is aligned by bowtie and RNA transcript expression estimated by RSEM. Tumor vs. matched-normal variant analysis was performed using the NantOmics Contraster analysis pipeline to determine somatic and germline SNVs, insertions and deletions, and identify highly amplified regions of the tumor genome.

Small variants were annotated with base-level PhastCons conservation scores, population allele frequencies from dbSNP (Build 142), and their predicted impact to gene transcripts downloaded from the RefSeq database (e.g., changes in DNA sequence and protein).

Identification of Tumor Somatic Single Nucleotide Variants (SNVs):

Whole-genome DNA sequencing of 45 lung cancer patients' tumor and normal (germline) genomes resulted in the identification of 802 missense or nonsense protein-altering SNVs in the panel of 35 genes associated with lung cancer etiology. The panel included 25 genes considered somatic tumor drivers (tumor driver gene panel), and 10 genes known to affect inherited cancer risk (inherited risk gene panel; Table 1). Among the 45 lung cancer patients, the total of 802 SNVs occurred at 147 unique SNV sites. All 802 variants were present in the tumor genomes. Bioinformatic analysis of tumor and normal germline DNA sequence showed that 701 of the 746 SNVs (94%) originated in the germline, and the remaining 45 SNVs (6%) originated in somatic tissue. Applying the same gene panel to the analysis of 621 cancer patients' with 33 cancer types, tumor-normal sequencing analysis resulted in the identification of 10,704 missense or nonsense protein-altering SNVs. There were 919 unique SNVs sites that contributed to the 10,704 SNVs identified. Analysis of each patient's tumor and normal germline genome determined that 10,149 (95%) of the SNVs were of germline origin, while the remaining 555 (5%) SNVs were of somatic origin.

TABLE 1

| | Numbers of Variants in Patients with All | | | Numbers of Variants in Lung Cancer | | |
|---|---|---|---|---|---|---|
| | Cancer Types | | | Patients Only | | |
| Gene | Unique | Germline | Somatic | Unique | Germline | Somatic |

Tumor Driver Gene Panel

| ALK | 32 | 1317 | (99%) | 14 | (1%) | 6 | 93 | (99%) | 1 | (1%) |
|---|---|---|---|---|---|---|---|---|---|---|
| BRAF | 23 | 5 | (15%) | 29 | (85%) | 3 | 0 | (0%) | 3 | (100%) |
| CDKN2A | 22 | 35 | (71%) | 14 | (29%) | 5 | 2 | (40%) | 3 | (60%) |
| CEBPA | 8 | 2 | (25%) | 6 | (75%) | 0 | 0 | | 0 | |
| DNMT3A | 22 | 12 | (52%) | 11 | (48%) | 1 | 1 | (100%) | 0 | (0%) |
| EGFR | 29 | 315 | (95%) | 16 | (5%) | 6 | 15 | (71%) | 6 | (29%) |
| ERBB2 | 38 | 921 | (98%) | 15 | (2%) | 7 | 68 | (100%) | 0 | (0%) |
| EZH2 | 12 | 117 | (94%) | 8 | (6%) | 1 | 3 | (100%) | 0 | (0%) |
| FLT3 | 25 | 846 | (99%) | 5 | (1%) | 6 | 64 | (98%) | 1 | (2%) |
| IDH1 | 9 | 85 | (94%) | 5 | (6%) | 2 | 2 | (100%) | 0 | (0%) |
| IDH2 | 10 | 9 | (64%) | 5 | (36%) | 0 | 0 | | 0 | |
| JAK2 | 18 | 37 | (88%) | 5 | (12%) | 0 | 0 | | 0 | |
| KIT | 19 | 138 | (93%) | 10 | (7%) | 5 | 8 | (62%) | 5 | (38%) |
| KMT2A | 57 | 72 | (80%) | 18 | (20%) | 3 | 2 | (67%) | 1 | (33%) |
| KRAS | 16 | 3 | (4%) | 77 | (96%) | 4 | 0 | (0%) | 7 | (100%) |
| MET | 28 | 58 | (84%) | 11 | (16%) | 5 | 7 | (87%) | 1 | (13%) |
| NOTCH1 | 59 | 143 | (89%) | 17 | (11%) | 8 | 6 | (75%) | 2 | (25%) |
| NPM1 | 2 | 1 | (50%) | 1 | (50%) | 0 | 0 | | 0 | |
| NRAS | 10 | 1 | (5%) | 18 | (95%) | 0 | 0 | | 0 | |
| PDGFRA | 24 | 169 | (92%) | 14 | (8%) | 2 | 9 | (100%) | 0 | (0%) |
| PDGFRB | 28 | 98 | (92%) | 8 | (8%) | 8 | 11 | (92%) | 1 | (8%) |
| PGR | 31 | 377 | (96%) | 15 | (4%) | 7 | 21 | (91%) | 2 | (9%) |
| PIK3CA | 31 | 96 | (54%) | 82 | (46%) | 2 | 6 | (86%) | 1 | (14%) |
| PTEN | 33 | 780 | (97%) | 24 | (3%) | 2 | 56 | (100%) | 0 | (0%) |
| RET | 22 | 244 | (96%) | 9 | (4%) | 7 | 21 | (100%) | 0 | (0%) |

| | | | | | | |
|---|---|---|---|---|---|---|
| Total | 608 | 5881 | 437 | 90 | 395 | 34 |

Inherited Risk Gene Panel

| | | | | | | | | | | |
|---|---|---|---|---|---|---|---|---|---|---|
| APC | 85 | 692 | (92%) | 58 | (8%) | 7 | 48 | (98%) | 1 | (2%) |
| BMPR1A | 5 | 334 | (99%) | 2 | (1%) | 1 | 17 | (100%) | 0 | (0%) |
| EPCAM | 13 | 464 | (100%) | 0 | (0%) | 3 | 37 | (100%) | 0 | (0%) |
| MLH1 | 15 | 295 | (99%) | 4 | (1%) | 4 | 26 | (96%) | 1 | (4%) |
| MSH2 | 23 | 40 | (89%) | 5 | (11%) | 4 | 5 | (100%) | 0 | (0%) |
| MSH6 | 25 | 273 | (98%) | 7 | (2%) | 2 | 18 | (100%) | 0 | (0%) |
| PMS2 | 44 | 1558 | (99%) | 10 | (1%) | 13 | 110 | (97%) | 3 | (3%) |
| POLD1 | 30 | 208 | (97%) | 7 | (3%) | 4 | 11 | (100%) | 0 | (0%) |
| POLE | 58 | 398 | (96%) | 18 | (4%) | 16 | 34 | (92%) | 3 | (8%) |
| STK11 | 13 | 6 | (46%) | 7 | (54%) | 3 | 0 | (0%) | 3 | (100%) |

| | | | | | | |
|---|---|---|---|---|---|---|
| Total | 311 | 4268 | 118 | 57 | 306 | 11 |

For lung cancer patients, just 7% and 3% of SNVs were of somatic origin in the tumor driver gene panel and inherited risk gene panels, respectively. Among all cancer patients, the percentage of SNVs representing somatic changes was 6% and 3% for genes in the tumor driver gene panel and inherited risk gene panel, respectively. A greater percentage of somatic variants was expected to be observed among the 25 genes that are known to harbor somatic cancer driver mutations. There was significant variation in the number of SNVs observed in each gene. The number of unique SNV sites was strongly correlated with the size of the gene protein-coding sequence (p-value<10-9, R2=0.70 for all cancer types). However, there was no correlation between the number of germline, somatic, or total variants and the size of the gene (all p-values>0.40). The degree of association between each gene and the cancer outcomes is a likely determinant of the variation in SNV counts observed between genes as well as the natural population genetic variation present in each gene. Furthermore, specific cancer driver SNVs are enriched among the patients.

The small number of unique variants compared to total variants illustrates the presence of common SNVs that are observed in many genomes in the study population of cancer patients. There were 21 variants that had allele frequencies >0.02 in the sample of 621 cancer patients, 17 of which were common germline SNPs and 4 of which were common somatic driver mutations (2 in KRAS and 2 in PIK3CA). All 21 common variants are archived in the single nucleotide polymorphism database (dbSNP) of genetic polymorphisms. Among all patients, 645 of the 919 total unique variants (70%) were observed only once. Three SNVs were of both germline and somatic origin.

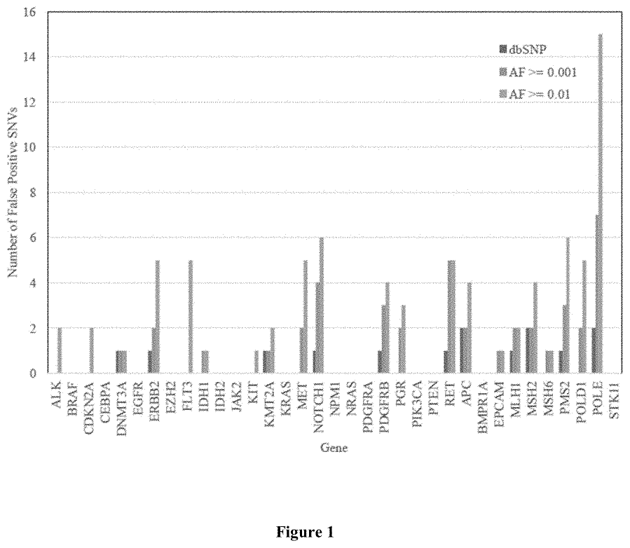

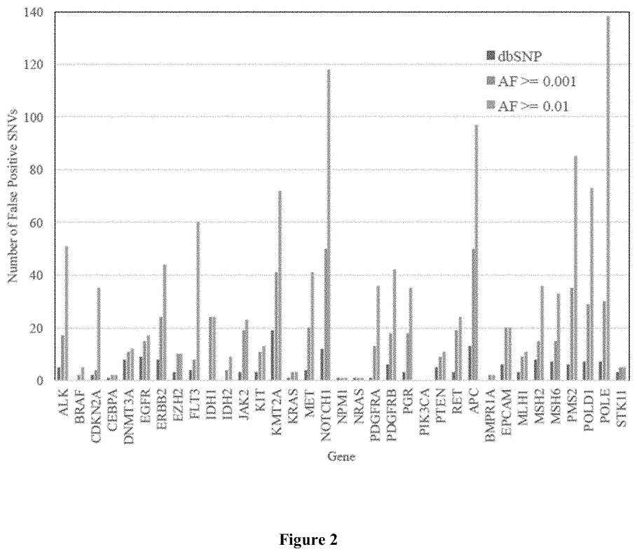

Tumor genome sequencing alone (without comparison to the normal germline genome) of the lung cancer patients would identify 746 missense and nonsense protein-altering SNVs (Table 1). In the context of tumor molecular profiling, any SNV of germline origin that is categorized as of somatic origin constitutes a false positive result. Without any filtering of putative germline variants, false positive rates of approximately 94% are expected, given the data presented in Table 1. FIG. 1 shows the number of false positive results that would occur among the 45 lung cancer patients and FIG. 2 depicts the same result for all 621 cancer patients for each gene with three different SNV filtering criteria: 1) removing all SNVs that are found in the dbSNP database; 2) removing all SNVs with reported population allele frequencies ≥0.01 (1%); and 3) removing all SNVs with reported population allele frequency ≥0.001 (0.1%). (An additional three SNVs that had no reported population allele frequencies but were common germline SNVs among the cancer patients and they were present in dbSNP were also removed). The largest numbers of false positive results occurred using an allele frequency threshold of 0.01. The number of false positives could be reduced by half in most genes by reducing the allele frequency filtering threshold to 0.001. The precision of most publicly-available population allele frequency estimates did not exceed 0.0001 so further reductions in the population allele frequency threshold had a nominal effect on the number of false positive SNVs.

Excluding all of the SNPs that were present in the dbSNP database resulted in the lowest numbers of false positive SNVs. However, the improved false positive rate came at the cost of an increased false negative rate, as many true tumor somatic SNVs were excluded. Excluding all SNVs present in dbSNP resulted in 17 false negatives among 45 true tumor somatic variants observed in the 45 lung cancer patients (38%), and 245 false negatives out of the 555 true somatic variants among the lung cancer patients (44%). Using the 0.001 allele frequency threshold filter, there were 41 false positive results (5% of the 746 total SNVs observed and 48% of the 86 SNVs remaining after filtering) and zero false negative results among lung cancer patients. The same filtering threshold resulted in 554 false positive results (5% of the 10,704 total SNVs observed and 50% of the 1,107 SNVs remaining after filtering) and zero false negative results among all 621 cancer patients.

Consequences of the Tumor-Only Sequencing Approach:

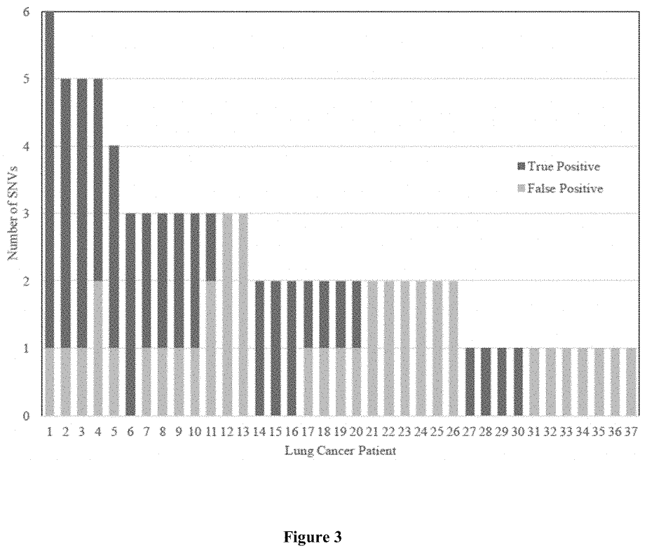

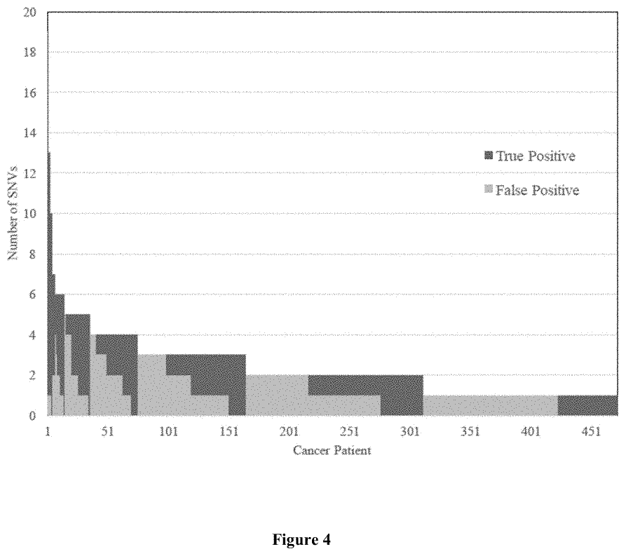

After filtering to remove all SNVs with a population allele frequency ≥0.001, 37 of the 45 lung cancer patients, and 472 of the 621 all cancer patients had at least one missense or nonsense protein-altering SNV in the panel of 35 genes. The 7 lung cancer and 149 total patients without SNVs after filtering did not have any true somatic variants, showing that the population allele frequency filter did not produce false negative results. FIG. 3 shows the number of true positive (i.e., the number of tumor somatic SNVs) and false positive SNVs (i.e., the number of inherited germline SNVs) for the lung cancer and FIG. 4 shows the same results for all patients that had at least one SNV remaining after filtering. The average numbers of SNVs were 1.91 and 1.84, for lung cancer and all cancer patients, respectively. One patient with 39 somatic SNVs was excluded from FIG. 2b for presentation purposes. In lung cancer patients, 29 of the 45 patients (65%) had at least one false positive SNV, and 15 patients had only false positive SNVs (33%), without any true positive results. While only 5% of the total SNVs found among lung cancer patients were false positives after filtering at a population allele frequency of 0.001 (41 false positives out of 802 total SNVs discovered), the SNVs were distributed across 65% of the patients. The majority of the 802 SNVs discovered are common variants that are excluded by filtering. These results highlight the impact of rare germline mutations on the rate of false positive discoveries. In the full study population, 365 of the 621 patients (59%) had at least one false positive SNV, yielding an average of 0.91 false positives per patient. Only false positive SNVs, without true positive results, were present in 193 of the 621 patients (31%).

False positive SNVs can have a direct detrimental impact on patient care. Table 2 shows 12 druggable genes, the specific drugs that target each of the genes when they are somatically mutated, and the number of patients with at least 1 false positive SNV observed in each of the genes. Furthermore, the cost and possible adverse health effects associated with each drug are shown to illustrate the financial and clinical implications of prescribing a drug based on a false positive result. Tumor-only sequence analysis can put patients at unnecessary risk of serious adverse drug effects, along with the negative impact of prescribing a drug treatment that is likely to be non-efficacious.

TABLE 2

| | | Number of Patients with at least one False | | | | | | Approximate | |
|---|---|---|---|---|---|---|---|---|---|
| | Gene | Positive Variant after Each SNV Filter | | | | | | Drug Cost | Warning and |
| | Targeted | No Filter | | AF >= 0.01 | | AF >= 0.001 | | per | Precautions |
| Drug | by Drug | All | LC | All | LC | All | LC | patients^{a} | (FDA Label) |
| Crizotinib | ALK | 621 | 45 | 50 | 2 | 16 | 0 | $18,349.50 | Pneumonitis, Hapatic |
| | | | | | | | | | Abnormalities, QT |
| | | | | | | | | | Prolongation |
| Alectinib | | | | | | | | $15,976.33 | Hepatotoxicity, |
| | | | | | | | | | ILD/Pneumonitis, |
| | | | | | | | | | Bradycardia, Myalgia, |
| | | | | | | | | | CPK elevation, EFT |
| Ceritinib | | | | | | | | $18,964.13 | GI toxicity, |
| | | | | | | | | | Hepatotoxicity, |
| | | | | | | | | | ILD/Pneumonitis, QT |
| | | | | | | | | | prolongation, |
| | | | | | | | | | Hyperglycemia, |
| | | | | | | | | | Bradycardia, |
| | | | | | | | | | Pancreatitis, EFT |
| Brigatinib | | | | | | | | $15,960.00 | ILD/Pneumonitis, HTN, |
| | | | | | | | | | Bradycardia, Visual |
| | | | | | | | | | disturbance, CPK |
| | | | | | | | | | elevation, Pancreatic |
| | | | | | | | | | enzyme elevation, |
| | | | | | | | | | Hyperglycemia, EFT |
| Vemurafenib | BRAF | 5 | 0 | 5 | 0 | 2 | 0 | $13,020.94 | Hypersensitivity, |
| | | | | | | | | | Dermatologic |
| | | | | | | | | | reactions, QT |
| | | | | | | | | | Prolongation, |
| | | | | | | | | | Hepatotoxicity, |
| | | | | | | | | | Ophthalmologic |
| | | | | | | | | | reactions, Renal |
| | | | | | | | | | failure, EFT |
| Dabrafenib | | | | | | | | $11,412.43 | Febrile drug reaction, |
| | | | | | | | | | Hyperglycemia, Uveitis |
| | | | | | | | | | and Iritis, G6PD |
| | | | | | | | | | deficiency, EFT |
| Cobimetinib | | | | | | | | $7,856.04^{a} | Hemorrhage, |
| | | | | | | | | | Cardiomyopathy, |
| | | | | | | | | | Dermatologic |
| | | | | | | | | | reactions, Retinopathy |
| | | | | | | | | | and RVO, |
| | | | | | | | | | Hepatotoxicity, |
| | | | | | | | | | Rhabdomyolysis, |
| | | | | | | | | | Photosensitivity, EFT |
| Trametinib | | | | | | | | $12,450.00 | Cardiomyopathy, |
| | | | | | | | | | RPED, RVO, ILD, Skin |
| | | | | | | | | | toxicity, EFT |
| Azacitidine | DNMT3A | 12 | 1 | 12 | 1 | 11 | 1 | $2,221.81^{c} | Cytopenias, |
| | | | | | | | | | Hepatotoxicity, Renal |
| | | | | | | | | | abnormalities, EFT |
| Decitabine | | | | | | | | $3,967.37^{c} | Cytopenias, EFT |
| Erlotinib | EGFR | 303 | 15 | 16 | 0 | 14 | 0 | $9,390.44 | ILD, Renal failure, |
| | | | | | | | | | Hepatotoxicity, GI |
| | | | | | | | | | perforations, Bullous |
| | | | | | | | | | and skin disorders, |
| | | | | | | | | | CVA, MAHA, Ocular |
| | | | | | | | | | disorders, EFT |
| Afatinib | | | | | | | | $9,060.85 | Diarrhea, Bullous and |
| | | | | | | | | | skin disorders, ILD, |
| | | | | | | | | | Hepatic toxicity, |
| | | | | | | | | | Keratitis, EFT |
| Gefitinib | | | | | | | | $9,117.36 | Diarrhea, Bullous and |
| | | | | | | | | | skin disorders, ILD, |
| | | | | | | | | | Hepatic toxicity, |
| | | | | | | | | | Keratitis, EFT, GI |
| | | | | | | | | | perforation |
| Neratinib | ERBB2 | 544 | 37 | 43 | 5 | 24 | 2 | $12,600.00 | Diarrhea, |
| | | | | | | | | | Hepatotoxicity, EFT |
| Lapitinib | | | | | | | | $6,314.31 | Decreased LVEF, |
| | | | | | | | | | Hepatotoxicity, |
| | | | | | | | | | Diarrhea, ILD and |
| | | | | | | | | | pneumonitis, QT |
| | | | | | | | | | interval prolongation, |
| | | | | | | | | | EFT |
| Ruxolitinib | JAK2 | 37 | 0 | 23 | 0 | 19 | 0 | $12,932.64 | Cytopenias, Infection |
| Imatinib | KIT | 135 | 8 | 13 | 1 | 11 | 0 | $23,152.39 | Edema, Cytopenias, |
| | | | | | | | | | CHF and LV |
| | | | | | | | | | dysfunction, |
| | | | | | | | | | Hepatotoxicity, |
| | | | | | | | | | Hemorrhage, GI |
| | | | | | | | | | perforations, |
| | | | | | | | | | Cardiogenic shock, |
| | | | | | | | | | Bullous, |
| | | | | | | | | | Hypothyroidism, EFT |
| Dasatinib | | | | | | | | $16,084.02 | Myelosuppression, |
| | | | | | | | | | Thrombocytopenia, |
| | | | | | | | | | Fluid retention, QT |
| | | | | | | | | | Prolongation, CHF, LV |
| | | | | | | | | | dysfunction, Ml, EFT |
| Regorafenib | | | | | | | | $17,857.80^{d} | Hemorrhage, |
| | | | | | | | | | Dermatological |
| | | | | | | | | | toxicity, HTN, Cardiac |
| | | | | | | | | | ischemia and |
| | | | | | | | | | infarction, RPLS, GI |
| | | | | | | | | | perforation, Wound |
| | | | | | | | | | healing complications, |
| | | | | | | | | | EFT |
| Crizotinib | MET | 58 | 7 | 41 | 5 | 20 | 2 | $18,349.50 | Pneumonitis, Hepatic |
| | | | | | | | | | Lab Abnormalities, QT |
| | | | | | | | | | Interval Prolongation, |
| | | | | | | | | | EFT |
| Cabozantinib | | | | | | | | $18,191.26 | Hemorrhage, GI |
| | | | | | | | | | perforations, |
| | | | | | | | | | Thrombotic events, |
| | | | | | | | | | HTN, Diarrhea, PPES, |
| | | | | | | | | | RPLS, EFT |
| Axitinib | PDGFRA | 160 | 9 | 36 | 0 | 13 | 0 | $16,416.28 | Hemorrhage, GI |
| | | | | | | | | | perforations, |
| | | | | | | | | | Thrombotic events, |
| | | | | | | | | | HTN, Hypothyroidism, |
| | | | | | | | | | RPLS, EFT |
| Regorafenib | | | | | | | | $17,857.80^{d} | Hemorrhage, |
| | | | | | | | | | Dermatological |
| | | | | | | | | | toxicity, HTN, Cardiac |
| | | | | | | | | | ischemia and |
| | | | | | | | | | infarcation, RPLS, GI |
| | | | | | | | | | perforation, Wound |
| | | | | | | | | | healing complications, |
| | | | | | | | | | EFT |
| Axitinib | PDGFRB | 89 | 9 | 42 | 4 | 18 | 3 | $16,416.28 | Hemorrhage, GI |
| | | | | | | | | | perforations, |
| | | | | | | | | | Thrombotic events, |
| | | | | | | | | | HTN, Hypothyroidism, |
| | | | | | | | | | RPLS, EFT |
| Regorafenib | | | | | | | | $17,857.80^{d} | Hemorrhage, |
| | | | | | | | | | Dermatological |
| | | | | | | | | | toxicity, HTN, Cardiac |
| | | | | | | | | | ischemia and |
| | | | | | | | | | infarcation, RPLS, GI |
| | | | | | | | | | perforation, Wound |
| | | | | | | | | | healing complications, |
| | | | | | | | | | EFT |
| Idelalisib | PIK3CA | 96 | 6 | 0 | 0 | 0 | 0 | $5,721.26^{e} | Cutaneous reactions, |
| | | | | | | | | | Anaphylaxis, |
| | | | | | | | | | Neutropenia, EFT |
| Everolimus | | | | | | | | $17,013.54 | Pneumonitis, |
| | | | | | | | | | Infections, Oral |
| | | | | | | | | | ulceration, EFT |
| Cabozantinib | RET | 217 | 18 | 22 | 5 | 19 | 5 | $18,191.26 | Hemorrhage, GI |
| | | | | | | | | | perforations, |
| | | | | | | | | | Thrombotic events, |
| | | | | | | | | | HTN, Diarrhea, PRES, |
| | | | | | | | | | RPLS, EFT |
| Vandetinib | | | | | | | | $15,445.43 | QT prolongation, Skin |
| | | | | | | | | | reactions, ILD, |
| | | | | | | | | | Ischemic |
| | | | | | | | | | cerebrovascular |
| | | | | | | | | | events, Hemorrhage, |
| | | | | | | | | | Diarrhea, HTN, RPLS, |
| | | | | | | | | | EFT |
| Total number | 621 | 45 | 303 | 23 | 167 | 13 | | | |
| of unique | (100%) | (100%) | (49%) | (51%) | (27%) | (29%) | | | |
| patients with | | | | | | | | | |
| a FP SNV | | | | | | | | | |

AF = population allele frequency; All = patients with all 30 cancer types; LC = lung cancer patients only; ILD = Interstitial lung disease; EFT = Embryofetal toxicity; RVO = Retinal vein occlusion; RPED = Retinal pigment epithelial dystrophy; CVA = Cerebrovascular accident; MAHA = Microangiopathic hemolytic anemia; GI = Gastrointestinal; LVEF = Left ventricular ejection fraction; MI = Myocardial infarction; RPLS = Reversible posterior leukoencephalopathy syndrome; PRES = Posterior reversible encephalopathy syndrome; HTN = Hypertension (including hypertensive crisis);
^{a}Average wholesale price for 30 days unless otherwise noted.
^{b}Drug not given continuously.
^{c}Single cycle based on body surface area of 2.02.
^{d}Based on 21 days on and 7 days off schedule.
^{e}Based on 14 days on and 14 days of schedule.

Expression of Somatic Single Nucleotide Variants: RNA sequencing data allowing assessment of the expression of the tumor somatic SNVs was available from 26 lung cancer patients and 378 of all patients. Table 3 shows the total number of somatic SNVs assessed, the number of somatic SNVs that were not expressed, and the number of patients with a somatic SNV that was not expressed. A significant percentage of SNVs were not expressed: 18% (7 out of 39 SNVs) for lung cancer patients, and 15% (75 out of 517 SNVs) for all cancer patients. There was substantial variation in the percent of expressed tumor somatic variants between genes. Nearly 80% or more of SNVs in FLT3, PDGFRA, PGR, and RET were not expressed among all cancer patients. In the study population, 9% of lung cancer patients (6 of all 26 patients with tumor RNA sequencing data) and 13% of all cancer patients (51 of 378 total cancer patients with tumor RNA sequencing data) had at least one true tumor somatic SNV that was not expressed in the messenger RNA. There were 4 tumor somatic SNVs in 4 lung cancer patients that were not expressed in the twelve genes that are targets for specific drugs shown in Table 2. There were 33 of all cancer patients with tumor somatic SNVs that were not expressed in the RNA. Treatment decisions based on DNA analysis alone might thus result in administration of ineffective therapies.

TABLE 3

| | All Cancer Types | | | | Lung Cancer Only | | | |
|---|---|---|---|---|---|---|---|---|
| | | Somatic SNVs | | Patients with | | Somatic SNVs | | Patients with |
| | Somatic | Not Expressed | | Not Expressed | Somatic | Not Expressed | | Not Expressed |
| Gene | SNVs | (%) | | SNV | SNVs | (%) | | SNV |
| ALK | 13 | 10 | (76%) | 9 | 0 | 0 | | 0 |
| BRAF | 24 | 0 | (0%) | 0 | 2 | 0 | (0%) | 0 |
| CDKN2A | 13 | 2 | (15%) | 2 | 3 | 0 | (0%) | 0 |
| CEBPA | 5 | 1 | (20%) | 1 | 0 | 0 | | 0 |
| DNMT3A | 11 | 1 | (9%) | 1 | 0 | 0 | | 0 |
| EGFR | 16 | 1 | (6%) | 1 | 6 | 0 | (0%) | 0 |
| ERBB2 | 14 | 1 | (7%) | 1 | 0 | 0 | | 0 |
| EZH2 | 8 | 0 | (0%) | 0 | 0 | 0 | | 0 |
| FLT3 | 5 | 4 | (80%) | 4 | 1 | 1 | (100%) | 1 |
| IDH1 | 5 | 0 | (0%) | 0 | 0 | 0 | | 0 |
| IDH2 | 5 | 0 | (0%) | 0 | 0 | 0 | | 0 |
| JAK2 | 5 | 1 | (20%) | 1 | 0 | 0 | | 0 |
| KIT | 8 | 5 | (63%) | 5 | 4 | 2 | (50%) | 2 |
| KMT2A | 18 | 2 | (11%) | 2 | 1 | 0 | (0%) | 0 |
| KRAS | 70 | 2 | (3%) | 2 | 6 | 1 | (17%) | 1 |
| MET | 11 | 3 | (27%) | 3 | 1 | 1 | (100%) | 1 |
| NOTCH1 | 16 | 1 | (6%) | 1 | 2 | 0 | (0%) | 0 |
| NPM1 | 1 | 0 | (0%) | 0 | 0 | 0 | | 0 |
| NRAS | 15 | 0 | (0%) | 0 | 0 | 0 | | 0 |
| PDGFRA | 14 | 11 | (79%) | 8 | 0 | 0 | | 0 |
| PDGFRB | 8 | 3 | (38%) | 3 | 1 | 1 | (100%) | 1 |
| PGR | 14 | 13 | (93%) | 11 | 1 | 1 | (100%) | 1 |
| PIK3CA | 75 | 0 | (0%) | 0 | 1 | 0 | (0%) | 0 |
| PTEN | 23 | 1 | (4%) | 1 | 0 | 0 | | 0 |
| RET | 9 | 7 | (78%) | 6 | 0 | 0 | | 0 |
| APC | 54 | 4 | (7%) | 4 | 1 | 0 | (0%) | 0 |
| BMPR1A | 1 | 0 | (0%) | 0 | 0 | 0 | | 0 |
| EPCAM | 0 | 0 | | 0 | 0 | 0 | | 0 |
| MLH1 | 4 | 0 | (0%) | 0 | 1 | 0 | (0%) | 0 |
| MSH2 | 5 | 0 | (0%) | 0 | 0 | 0 | | 0 |
| MSH6 | 7 | 1 | (14%) | 1 | 0 | 0 | | 0 |
| PMS2 | 10 | 0 | (0%) | 0 | 3 | 0 | (0%) | 0 |
| POLD1 | 7 | 0 | (0%) | 0 | 0 | 0 | | 0 |
| POLE | 16 | 1 | (6%) | 1 | 2 | 0 | (0%) | 0 |
| STK11 | 7 | 0 | (0%) | 0 | 3 | 0 | (0%) | 0 |
| Total | 517 | 75 | (15%) | 51 unique | 39 | 7 | (18%) | 6 unique |

Currently, two sequencing-based approaches are available to identify a patient's tumor somatic variation. In the first approach, the tumor DNA representing a targeted gene panel, the exome, or whole genome is sequenced, and putative germline variation is filtered based on a reference genome and the characteristics of the individual genomic variants discovered in the tumor (termed tumor-only analysis). Identification of a genomic variant in a population genetic database at an appreciable allele frequency is a common filtering criterion for determining if a variant is of inherited germline origin. The second and more precise approach as shown herein, is to use the patient's own germline genome as the precise control (rather than a reference genome for filtration) for distinguishing the inherited germline variants from those that are somatically derived (termed tumor-normal analysis). The currently CMS approved test for informing lung cancer treatment is based on the former approach and specifically excludes the use of normal tissue (germline information) in determining somatic variants.

In contrasting the two approaches, the inventors analyzed tumor and normal DNA sequencing data from 45 lung cancer and 621 total cancer patients versus a tumor only gene panel approved for coverage by CMS. The study demonstrated a 94% false positive rate (95% for all cancers) when using tumor-only sequencing to identify somatic variants. Even after utilizing multiple methods for bioinformatically filtering polymorphisms from the putative somatic mutations, the false positive rates still ranged from 38%-94%. Depending on the method used, excessively stringent filtering led to potential false negatives. When focusing on a subset of 12 genes targeted by FDA-approved drugs, where identification of somatic mutations could inform treatment decisions, the percentage of lung cancer patients affected by false positive calls ranged from 29%-51% depending on the method of polymorphism filtration used. Further risk of false positive results stem from the identification of variants identified from somatic tissue, i.e., true somatic mutations misidentified as deleterious (inherited) germline variants in such genes as BRCA1, BRCA2, and ATM. In 10 genes associated with germline risk for familial disease (the inherited risk gene panel), true somatic mutations in germline genes were discovered in 10 lung cancer patients (11 variants) and 101 total patients (118 variants) when using the tumor-only sequencing approach.

Sequencing and analysis of data from the patient's normal germline genome and tumor genome eliminates false positive results associated with analysis of tumor genome sequence data alone. The potential for tumor somatic SNVs to fruitfully inform patient treatment depends on expression of the DNA variants as messenger RNA, and then translation into protein. RNA sequencing of the tumor provides valuable information about relative expression levels of cancer driver genes, and the gene expression of specific tumor somatic variants. RNA expression analysis in this study showed that 18% of true somatic mutations identified from tumor/normal sequencing of lung cancer patients, as well as 15% for all cancer patients, were not expressed at the level of messenger RNA. In the study population, these results could impact clinical decision making for 9% of lung cancer patients, and 13% of all cancer patients. The results presented herein provide further evidence of the advantages associated with heightened precision of molecular analysis for drug targeting derived from tumor/normal DNA sequencing plus RNA sequencing.

In view of the above, it should therefore be appreciated that simultaneous sequencing and bioinformatics analysis of the DNA of both the normal germline genome and the tumor genome is necessary for accurate identification of molecular targets for cancer therapy. Analysis of only the tumor genome results in a high false positive rate in SNV identification. Even higher precision is achieved with simultaneous tumor-normal DNA and RNA sequencing analysis. Treatment decisions based on tumor-only DNA analysis or in the absence of RNA analysis might result in administration of ineffective therapies while also increasing risk of negative drug-related side effects. When used to guide clinical decision-making, the approach of tumor-only gene-panel analysis may increase risk to patients, cause potential long-term negative health consequences, and increase healthcare costs.

### Example 2

In this example, the inventors included 204 cancer patients with 11 gastrointestinal (GI) cancer types with whole genome sequencing of both tumor and normal genomes. True positive (true somatic variants) and false positive (true germline variants estimated to be somatic variants) rates were measured for missense and nonsense single nucleotide variants (SNVs) in a 45-gene panel as shown below. The 45-gene panel included 26 known somatic driver genes, 14 inherited cancer risk genes, and 5 of these genes can act both as somatic tumor drivers and inherited risk genes. RNA sequencing was available for 139 of the 204 patients. Sequence alignment and SNV variant calling was performed using well-established and published bioinformatics methods. In preferred methods BAMBAM was used to synchronously and incrementally align and identify SNV using DNA and RNA sequences.

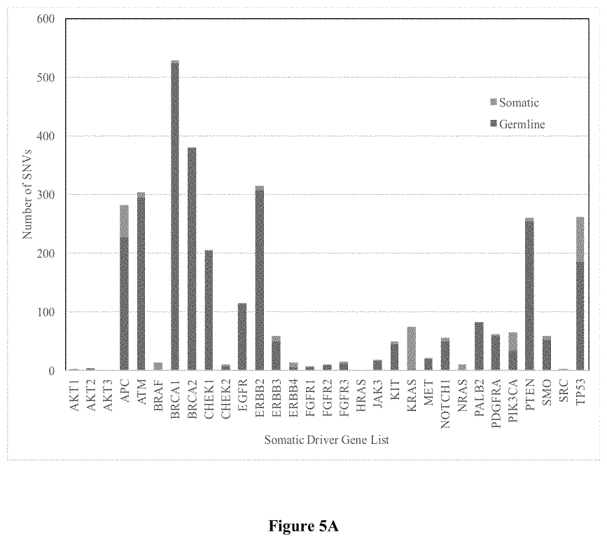

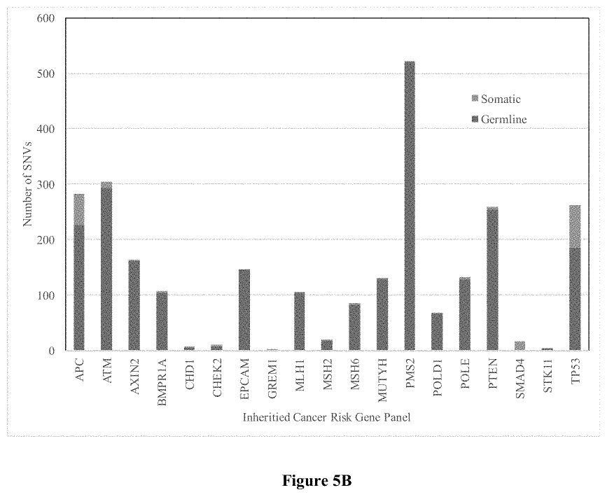

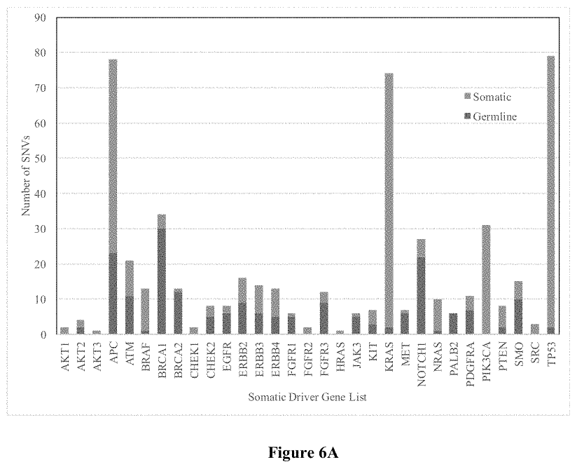

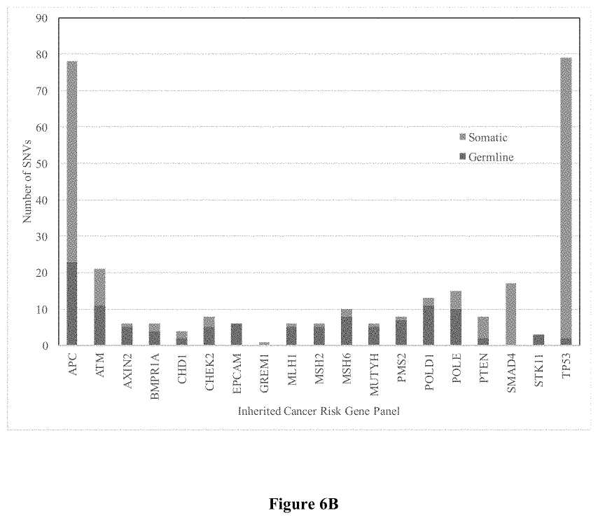

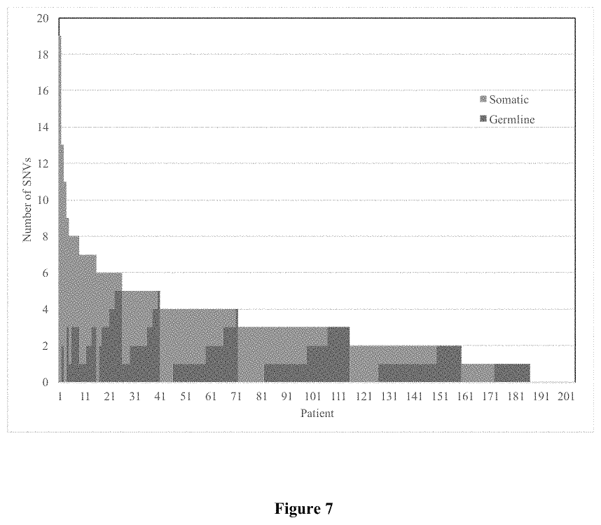

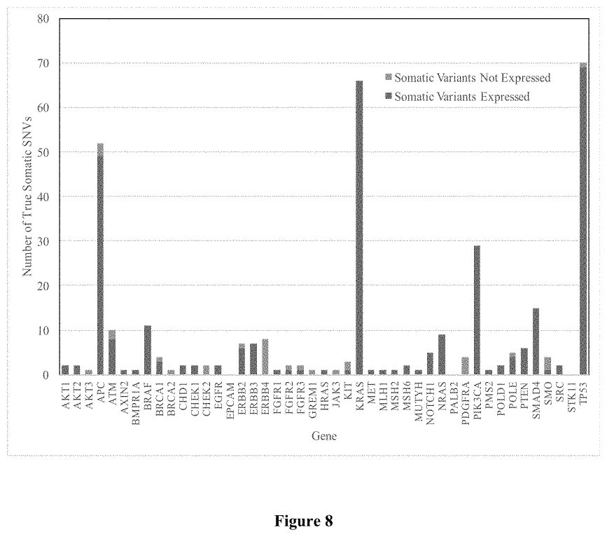

Results: 92% of SNVs identified from sequencing tumor genomes alone were of germline origin and potential false positives rather than true somatic variants (Somatic=true somatic variants; Germline=true germline variants). See FIGS. 5A and 5B. Notably, filtering all SNVs using public databases with reported population allele frequencies ≥0.001 still resulted in a false positive rate of 41% (Somatic=true somatic variants; Germline=true germline variants). See FIGS. 6A and 6B. 71% of GI patients had at least one false positive SNV (germline) after filtering on allele frequency (Somatic=true somatic variants; Germline=true germline variants) as is shown in FIG. 7. Moreover, RNA analysis showed that 10% of true somatic variants were not expressed and 17% of patients had at least one true somatic variant that was not expressed as can be taken from FIG. 8.

It should therefore be appreciated that sequencing the tumor genome identified all of the SNVs of inherited germline origin and tumor somatic origin, with the large majority being of germline origin. While population allele frequencies and other parameters could be used to filter SNV data and estimate somatic versus germline origin, such filtering was not accurately enough for clinical use. Further, it should be appreciated that simultaneous sequencing and bioinformatics analysis of DNA of both the normal germline genome and tumor genome is necessary for accurate identification of molecular targets. Analysis of tumor genome alone results in false-positive results. Higher precision is achieved with simultaneous tumor-normal DNA and tumor RNA sequencing analysis. Treatment decisions based on tumor-only DNA analysis or in the absence of RNA might result in administration of ineffective therapies while also increasing risk of negative drug-related side effects.

### Example 3

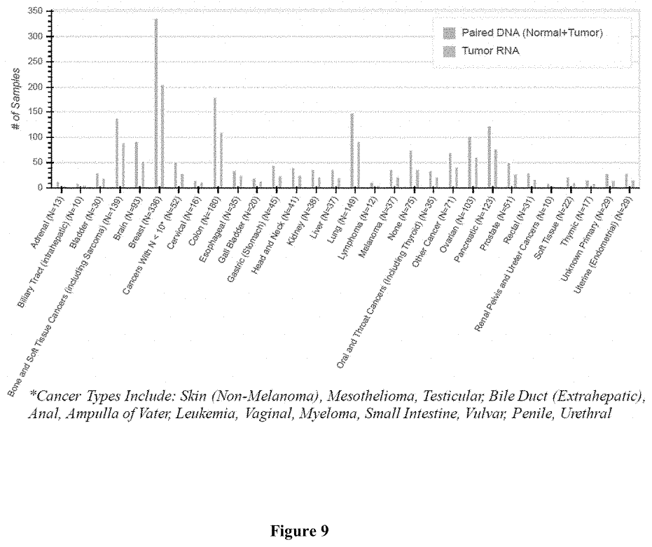

In this example, the inventors aimed to compare the accuracy and precision of tumor somatic calling with a 50 gene commonly used hotspot panel and analyzing the tumor tissue alone versus analyzing tumor DNA simultaneously with normal germline DNA and tumor RNA. Specifically, in this example, tumor samples and matched normal samples from 1879 cancer patients with 42 cancer types were obtained and whole genome sequencing data or whole exome sequencing data of those tissues were generated. The demographic overview of cohort is shown in Table 4 below, and the number of analytes sequenced by different cancer types are shown in FIG. 9 (the number of samples sequenced for DNA and/or RNA). Cancer with N<10 in Table 4 (or other cancer type in FIG. 9) includes skin (non-melanoma), mesothelioma, testicular, bile duct (extrahepatic), anal, ampulla of vater, leukemia, vaginal, myeloma, small intestine, vulvar, penile, urethral cancers.

TABLE 4

| | | | | Min. | Max | Median |
|---|---|---|---|---|---|---|
| Cancer Type | # Patients | # Male | # Female | Age | Age | Age |
| Breast | 336 | 2 | 327 | 20 | 86 | 56 |
| Colon | 180 | 83 | 93 | 17 | 87 | 38 |
| Lung | 149 | 67 | 78 | 9 | 90 | 65 |
| Bone and Soft Tissue Cancers | 139 | 72 | 62 | 0 | 82 | 49 |
| (including Sarcoma) | | | | | | |
| Pancreatic | 123 | 69 | 48 | 3 | 87 | 63 |
| Ovarian | 103 | 0 | 96 | 25 | 86 | 58 |
| Brain | 93 | 52 | 37 | 0 | 79 | 49 |
| Cancer Type Unknown | 75 | 38 | 29 | 6 | 91 | 59 |
| Other Cancer | 71 | 39 | 31 | 1 | 83 | 62 |
| Cancers With N < 10 [symbol] | 52 | 29 | 20 | 0 | 87 | 65.5 |
| Prostate | 51 | 48 | 0 | 40 | 83 | 65 |
| Gastric (Stomach) | 45 | 26 | 19 | 15 | 85 | 61 |
| Head and Neck | 41 | 31 | 8 | 19 | 86 | 64 |
| Kidney | 38 | 23 | 11 | 0 | 72 | 62 |
| Liver | 37 | 25 | 11 | 9 | 77 | 63 |
| Melanoma | 37 | 24 | 12 | 29 | 87 | 64 |
| Oral and Throat Cancers | 35 | 21 | 13 | 42 | 83 | 63 |
| (Including Thyroid) | | | | | | |
| Esophageal | 35 | 24 | 10 | 46 | 86 | 64 |
| Rectal | 31 | 21 | 10 | 28 | 80 | 57 |
| Bladder | 30 | 17 | 12 | 49 | 92 | 72 |
| Unknown Primary | 29 | 11 | 18 | 29 | 83 | 57 |
| Uterine (Endometrial) | 29 | 0 | 26 | 34 | 89 | 66 |
| Soft Tissue | 22 | 15 | 7 | 2 | 80 | 18 |
| Gall Bladder | 20 | 7 | 13 | 39 | 87 | 65.5 |
| Thy [symbol] | 17 | 9 | 8 | 24 | 75 | 59 |
| Cervical | 16 | 0 | 16 | 27 | 75 | 49 |
| Adrenal | 13 | 5 | 4 | 1 | 74 | 48 |
| Lymphoma | 12 | 5 | 3 | 18 | 81 | 66 |
| Renal Pelvis and | 10 | 5 | 5 | 8 | 71 | 42 |
| Ureter Cancers | | | | | | |
| Biliary tract | 10 | 5 | 4 | 46 | 78 | 61 |
| (intrahepatic) | | | | | | |

[symbol]

indicates data missing or illegible when filed

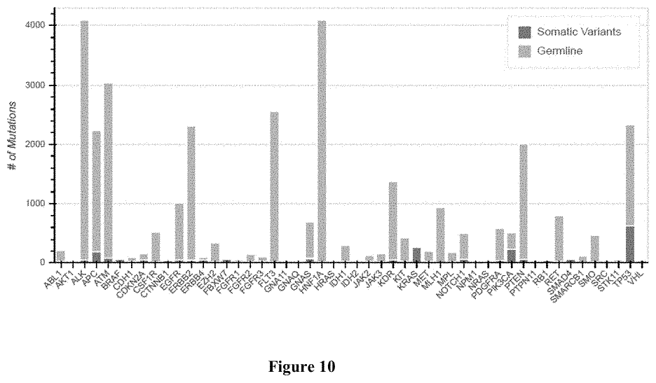

From the genomic sequencing data of the tumor tissue, the inventors determined that all patients have a least one germline single nucleotide variant (30955 single nucleotide variants total). Then, the inventors quantified the number of all single nucleotide variants (including those of germline origin and those of tumor somatic origin) identified from comparing the genomic sequencing data of the tumor and matched normal. 1127 out of 1879 patients (65%) had at least 1 somatic single nucleotide variants (308721 total). 741 out of 1135 (65%) of patients whose analytes were analyzed for paired DNA/RNA had at least 1 somatic single nucleotide variants (198844 total), resulting in 1775 unique single nucleotide variants amongst patients of paired DNA/RNA analysis. As shown in FIG. 10, 92% of single nucleotide variants identified from sequencing tumor genome alone were of germline origin, indicating that the majority of the single nucleotide variants identified from sequencing tumor genome alone can potentially be false positives rather than true somatic variants.

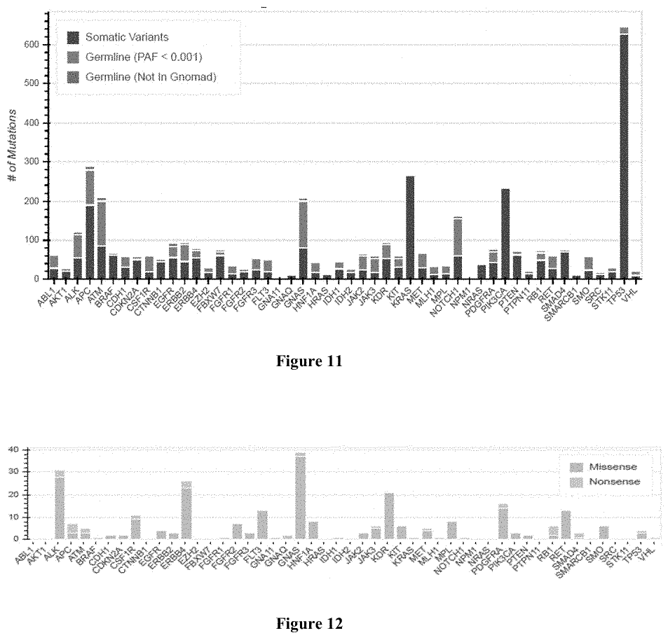

The inventors further filtered the identified single nucleotide variants from sequencing tumor genome alone using population allele frequencies and other parameters (e.g., known germline variants, gnomAD) to determine the ratio of single nucleotide variants (germline origin versus tumor somatic origin). As shown in FIG. 11, all single nucleotide variants identified from sequencing tumor genome alone were filtered using gnomAD with reported allele frequencies ≥0.001. The inventors found that the false positive rate after filtering is reduced to 34%. Yet, the inventors contemplate that such false positive rate is not sufficiently accurate for any clinical use of such data.

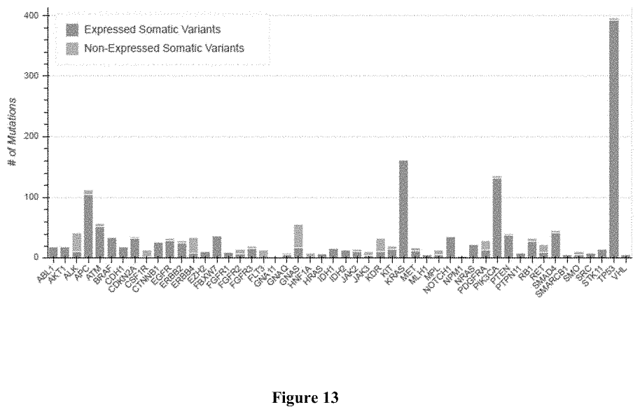

Further, the inventors found that not all single nucleotide variants of tumor somatic origin is expressed in RNA, indicating further filtering using RNA expression analysis is necessary to obtain the true somatic single nucleotide variants among all identified single nucleotide variants. As shown in FIG. 12 and FIG. 13, 15% of missense/nonsense somatic single nucleotide variants (shown in FIG. 12) and 17% of all somatic single nucleotide variants (missense/nonsense/synonymous) are not expressed. In addition, the inventors found that 23% of cancer patients in this example possessed at least one somatic single nucleotide variants (nonsense/missense) that are not expressed. From such data, the inventors contemplate that simultaneous sequencing and bioinformatics analysis of DNA, both the normal germline genome and tumor genome, is necessary for accurate identification of molecular targets as analysis of tumor genome alone results in high false-positive somatic variant calls and as lack of RNA expression may contribute less than clinical benefit in using the identified single nucleotide variants or genes having single nucleotide variants as molecular target. Viewed from different perspective, higher precision in identifying the tumor treatment and/or druggable target among genes and/or improved testing algorithm of tumor status can be achieved with simultaneous sequencing and bioinformatics analysis of DNA, both the normal germline genome and tumor genome.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

1. A method of performing a single nucleotide variant-based cancer test with increased accuracy, the method comprising:
determining presence of DNA single nucleotide variants in sequencing data derived from a tumor sample obtained from the patient relative to sequencing data derived from a matched normal patient sample;
determining expression of the DNA single nucleotide variants using RNA sequencing data derived from the tumor sample; and
identifying at least one DNA single nucleotide variant as associated with cancer status of the patient based on the presence and the expression of the single nucleotide variants.

2. The method of claim 1, wherein the DNA sequencing data is whole genome DNA sequencing data.

3. The method of claim 1, wherein the DNA sequencing data of the tumor tissue have a read depth of at least 50×.

4. The method of claim 1, wherein the DNA sequencing data of the matched normal tissue have a read depth of at least 30×.

5. The method of claim 1, wherein the step of determining the presence of the DNA single nucleotide variant is performed using location guided synchronous alignment of the DNA sequencing data from the tumor sample and the matched normal sample.

6. The method of claim 1, further comprising filtering the DNA single nucleotide variants using allele frequencies of the DNA single nucleotide variants.

7. The method of claim 1, wherein the DNA sequencing data of the tumor tissue have a read depth of at least 50×.

8. The method of claim 1, wherein the DNA sequencing data of the matched normal tissue have a read depth of at least 30×.

9. The method of claim 1, wherein the step of determining the presence of the DNA single nucleotide variant is performed using location guided synchronous alignment of the DNA sequencing data from the tumor sample and the matched normal sample.

10. The method of claim 1, further comprising filtering the DNA single nucleotide variants using allele frequencies of the DNA single nucleotide variants.

11. A method of identifying a treatment option for a patient with increased accuracy, the method comprising:
determining presence of DNA single nucleotide variants in the tumor sample relative to the matched normal sample of the patient;
determining expression of the DNA single nucleotide variants using the RNA sequencing data; and
identifying the treatment option targeting a gene having at least one DNA single nucleotide variant that is expressed as RNA.

12. The method of claim 11, wherein the determining the presence of the DNA single nucleotide variant is performed using location guided synchronous alignment of the DNA sequencing data from the tumor sample and the matched normal sample.

13. The method of claim 11, wherein the determining the presence of the DNA single nucleotide variant is performed using an in silica gene panel having a plurality of reference sequences of tumor associated genes.

14. The method of claim 11, wherein the determining the presence of the DNA single nucleotide variant is performed using an in silica gene panel having a plurality of reference sequences of tumor associated genes

15. The method of claim 13, wherein the in silica gene panel is cancer type-specific.

16. The method of claim 13, wherein the in silica gene panel is cancer type-specific.

17. The method of claim 13, wherein the tumor associated genes are selected from a group consisting of ABL1, EGFR, GNAS, KRAS, PTPN11, AKT1, ERBB2, GNAQ, MET, RBI, ALK, ERBB4, HNFIA, MLH1, RET, APC, EZH2, HRAS, MPL, SMAD4, ATM, FBXW7, IDH1, NOTCH1, SMARCB1, BRAF, FGFR1, JAK2, NPM1, SMO, CDH1, FGFR2, JAK3, NRAS, SRC, CDK 2A, FGFR3, IDH2, PDGFRA, STK11, CSF1R, FLT3, KDR, PIK3CA, TP53, CTN B1, GNA11, KIT, PTEN, VHL.

18. The method of claim 13, wherein the tumor associated genes are selected from a group consisting of ABL1, EGFR, GNAS, KRAS, PTPN11, AKT1, ERBB2, GNAQ, MET, RBI, ALK, ERBB4, HNF1A, MLH1, RET, APC, EZH2, HRAS, MPL, SMAD4, ATM, FBXW7, IDH1, NOTCH1, SMARCB1, BRAF, FGFR1, JAK2, NPM1, SMO, CDH1, FGFR2, JAK3, NRAS, SRC, CDKN2A, FGFR3, IDH2, PDGFRA, STK11, CSF1R, FLT3, KDR, PIK3CA, TP53, CTNNB1, GNA11, KIT, PTEN, VHL.

19. The method of claim 11, further comprising filtering the DNA single nucleotide variants using allele frequencies of the DNA single nucleotide variants.

20. The method of claim 11, further comprising filtering the DNA single nucleotide variants using allele frequencies of the DNA single nucleotide variants.

21-27. (canceled)
